(19) United States
(12) Patent Application Publication
(10) Pub. No.: US 2020/0197547 A1
(43) Pub. Date: Jun. 25, 2020

(54) ANTI-MESOTHELIN RADIOLABELLED SINGLE DOMAIN ANTIBODIES SUITABLE FOR THE IMAGING AND TREATMENT OF CANCERS

(71) Applicants: INSERM (Institut National de la Santé et de la Recherche Médicale), Paris (FR); Université Grenoble Alpes, Saint Martin d'Heres (FR); Centre Hospitalier Universitaire de Grenoble, Grenoble Cedex 09 (FR)

(72) Inventors: Alexis BROISAT, La Tronche (FR); Catherine GHEZZI, La Tronche (FR); Christopher MONTEMAGNO, La Tronche (FR); Daniel FAGRET, La Tronche (FR)

(21) Appl. No.: 16/642,185

(22) PCT Filed: Aug. 29, 2018
(86) PCT No.: PCT/EP2018/073174

§ 371 Date: Feb. 26, 2020

(30) Foreign Application Priority Data
Aug. 30, 2017 (EP) 17306110.2

(51) Int. Cl. A61K 51/10; C07K 16/30; A61P 35/04

(52) U.S. Cl. CPC A61K 51/1045; A61P 35/04; C07K 16/30; A61K 51/1093

(57) ABSTRACT

Mesothelin (MSLN) has been found to be overexpressed in several human malignancies: 100% of epithelial mesotheliomas, the majority of pancreatic and ovarian adenocarcinomas, more than 50% of lung adenocarcinomas and 34 to 67% of triple negative breast cancer (TNBC). The limited expression of mesothelin in normal human tissues and its overexpression in several aggressive human cancers make MSLN an attractive candidate for therapy. The objective of the inventors was to perform the nuclear imaging of TNBC xenografts with anti-MSLN single domain antibodies radiolabeled with ^{99m}Tc (^{99m}Tc-A1 and ^{99m}Tc-C6). They showed that ^{99m}Tc-A1 represent a good candidate for targeting mesothelin positive tumors. Accordingly, the present invention to an anti-mesothelin single domain antibody which is labelled with a radionuclide and its uses for imaging and/or treating cancer.

### FIELD OF THE INVENTION

The present invention relates to anti-mesothelin radio labelled single domain antibodies suitable for the imaging and treatment of cancers.

### BACKGROUND OF THE INVENTION

Triple Negative Breast Cancer (TNBC) is an invasive breast carcinoma that lacks expression of estrogen receptor (ER), human epidermal growth factor receptor 2 (HER2) and progesterone receptor (PR). About 10-20% of breast cancers are found to be TNBC. Patients with TNBC have an aggressive clinical course, with a high recurrence rate and short survival outcomes [1]. This breast cancer subtype lacks effective targeted therapies, and efforts are focused on the identification of new potential targets, such as the cell surface glycoprotein mesothelin (MSLN)[2,3]. MSLN constitutive expression is restricted to mesothelial cells lining the pericardium, peritoneum and pleura. The MSLN gene encodes a precursor protein of 71 kDa, processed into a shedded form (MPF: Megakaryocyte Potentiating Factor) and a 40 kDa membrane bound protein, mesothelin [4]. The biological function of MSLN is not well known, and no detectable abnormalities was observed in MSLN deficient mice [5]. However, MSLN has been found to be overexpressed in several human malignancies: 100% of epithelial mesotheliomas, the majority of pancreatic and ovarian adenocarcinomas, more than 50% of lung adenocarcinomas and 34 to 67% of TNBC [6,7,8,9]. MSLN seems to be involved in tumor aggressiveness since its expression has been correlated with a poorer patient outcome in several human cancers [10,11,12]. This might be attributed to MSLN induced metalloproteinases expression (MMP-7 and MMP-9) [13,14]. Moreover, in pancreatic cancer cell lines, MSLN overexpression resulted in increased Cyclin E and CDK2 expression, thereby promoting cell cycle progression and cell proliferation [15]. A role of MSLN has also been evoked in the resistance to Paclitaxel chemotherapy through the activation of the PI3K pathway [16]. In breast cancer, MSLN is associated with tumor infiltration into lymph node and a decrease of overall survival [17]. Among TNBC, patients with MSLN positive tumor developed more distant metastasis, and have lower overall and disease-free survival [18].

The limited expression of mesothelin in normal human tissues and its overexpression in several aggressive human cancers make MSLN an attractive candidate for therapy, including for TNBC [9,19]. Therefore, mesothelin targeted therapies are currently undergoing clinical trials [20]. Nevertheless, the identification of patients which could benefit of these MSLN targeting therapies remains challenging: Elevation of serum mesothelin has been shown in patients with mesothelioma [21,22] and ovarian cancer [23,24], but it has never been evaluated in breast cancer patients. Moreover, because of inter and intra-tumor heterogeneity, tumor phenotype cannot be accurately assessed by biopsy. Nuclear imaging is a highly sensitive non imaging modality that could address this challenge. Prantner et al. developed and characterized in vitro two anti-mesothelin single domain antibodies (sdAb), with high specificities and nanomolar affinities for the 40 kDa MSLN form [25].

### SUMMARY OF THE INVENTION

The present invention relates to anti-mesothelin (MSLN) radio labelled single domain antibodies suitable for the imaging and treatment of cancers. In particular, the present invention is defined by the claims.

### DETAILED DESCRIPTION OF THE INVENTION

The objective of the inventors was to perform the nuclear imaging of TNBC xenografts with the single domain antibodies radiolabeled with ^{99m}Tc (^{99m}Tc-A1 and ^{99m}Tc-C6). The inventors showed that ^{99m}Tc-A1 exhibited a high affinity for both MSLN (K_{D}=35 nM) as demonstrated in vitro on recombinant human protein and HCC70 cells and in vitro competition on HCC70 cells confirmed the specificity of this binding. ^{99m}Tc-C6 affinity for recombinant MSLN was 3-fold lower than that of ^{99m}Tc-A1 . ^{99m}Tc-A1 and ^{99m}Tc-C6 enabled non-invasive visualization of MSLN-positive tumors by SPECT imaging. High accumulation of ^{99m}Tc-A1 and ^{99m}Tc-C6 were observed in MSLN-positive HCC70 tumors whereas no signal was found in MSLN-negative MDA-MB-231 tumors. Moreover, ^{99m}Tc-A1 signal in HCC70 tumor was higher than that of ^{99m}Tc-C6. SPECT imaging quantification further confirmed those results. Indeed, ^{99m}Tc-A1 uptake was 5-fold higher in HCC70 tumors than in MDA-MB-231 tumors. Moreover, the in vivo competition study demonstrated the specificity of ^{99m}Tc-A1 binding to MSLN. Importantly, only minimal uptake was observed in the liver and intestine with ^{99m}Tc-A1 in comparison to ^{99m}Tc-C6. Taken together with its higher affinity and tumor uptake, this result suggests that ^{99m}Tc-A1 represent a good candidate for imaging and treating tumors.

The first object of the present invention relates to an anti-mesothelin single domain antibody which is labelled with a radionuclide wherein said single domain antibody i) binds to mesothelin with a KD of at least 5×10⁻⁸ M; and ii) cross-competes with the single domain antibody having the amino acid sequence SEQ ID NO:1 for binding to mesothelin.

As used herein, the term “mesothelin” or “MSLN” has its general meaning in the art and refers to a 40 kDa cell-surface glycosylphosphatidylinositol (GPI)-linked glycoprotein. The human mesothelin protein is synthesized as a 69 kD precursor which is then proteolytically processed. The 30 kD amino terminus of mesothelin is secreted and is referred to as megakaryocyte potentiating factor (Yamaguchi et al., J. Biol. Chem. 269:805 808, 1994). The 40 kD carboxyl terminus remains bound to the membrane as mature mesothelin (Chang et al., Natl. Acad. Sci. USA 93:136 140, 1996; Scholler et al., Cancer Lett 247(2007), 130-136). Exemplary nucleic acid and amino acid mesothelin sequences can be determined from the MSLN gene transcript found at NCBI accession number NM_005823 or NCBI accession number NM_013404.

As used herein the term “single domain antibody” has its general meaning in the art and refers to the single heavy chain variable domain of antibodies of the type that can be found in Camelid mammals which are naturally devoid of light chains. Such single domain antibody are also called VHH or “single domain antibody®”. For a general description of single domain antibodies, reference is made to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH domains given by the International ImMunoGeneTics information system aminoacid numbering (http://imgt.cines.fr/).

In some embodiments, the single domain antibody of the present invention binds to mesothelin with a dissociation constant (KD) of about 5×10⁻⁸ nM or less, about 45 nM or less, about 40 nM or less, about 35 nM or less, about 30 nM or less, about 25 nM or less, about 20 nM or less, or about 15 nM or less. In some embodiments, the dissociation constant is determined using surface plasmon resonance analysis, e.g., BIAcore analysis, according to standard methods known in the art. As used herein the term “surface plasmon resonance” includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see, e.g., Jonsson et al., Ann Biol Clin 51(1993), 19-26; Jonsson et al., Biotechniques 11(1991), 620-627; Johnsson et al., J Mol Recognit 8(1995), 125-131; and Johnnson et al., Anal Biochem 198(1991), 268-277. As used herein, the term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

Any competition assay known in the art or as described herein can be used to identify a single domain antibody that competes with any of the single domain antibodies described herein for binding to mesothelin. In some embodiments, such a competing single domain antibody binds to the same epitope (e.g., a linear or a conformational epitope) that is bound by a single domain antibody described herein. Methods for mapping the epitope to which an antibody or antibody-like molecule, e.g., a single domain antibody disclosed herein) binds are also known in the art, see, e.g., Morris, Epitope Mapping Protocols, in Methods in Molecular Biology vol. 66 (1996, Humana Press, Totowa, N.J.). In a non-limiting, exemplary competition assay, immobilized mesothelin is incubated in a solution comprising a first labeled single domain antibody that binds to mesothelin (e.g., as described herein) and a second unlabeled single domain antibody that is being tested for its ability to compete with the first single domain antibody for binding to mesothelin. As a control, immobilized mesothelin is incubated in a solution comprising the first labeled single domain antibody but not the second unlabeled single domain antibody. After incubation under conditions permissive for binding of the first single domain antibody to mesothelin, excess unbound single domain antibody is removed, and the amount of label associated with immobilized mesothelin is measured. If the amount of label associated with immobilized mesothelin is substantially reduced in the test sample relative to the control sample, then that indicates that the second single domain antibody competes with the first (or reference) single domain antibody for binding to mesothelin; see, e.g.,. Harlow and Lane (1988) Antibodies: A Laboratory Manual ch. 14 (Cold Spring Harbor Laboratory, Cold Spring Harbor, N.Y.).

In some embodiments, the single domain antibody of the present invention comprises (a) a CDR1 having a sequence set forth as SEQ ID NO:2 (GIDLSLYR), (b) a CDR2 having a sequence set forth as SEQ ID NO:3 (ITDDGTS); and (c) a CDR3 having a sequence set forth as SEQ ID NO:4 (NAETPLSPVNY).

In some embodiments, the single domain antibody of the present invention comprises an amino acid sequence having at least 70% of identity with SEQ ID NO:1. According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with the second amino acid sequence. Amino acid sequence identity is typically determined using a suitable sequence alignment algorithm and default parameters, such as BLAST P (Karlin and Altschul, 1990).

SEQ ID NO: 1:
Sequence of A1 single domain antibody FR1_CDR1_
FR2_CDR2_FR3-CDR3_FR4.
QVQLVQSGGGLVHPGGSLRLSCAASGIDLSLYRMRWYRQAPGKERDLVAL
ITDDGTSYYEDSVKGRFTITRDNPSNKVFLQMNSLKPEDTAVYYCNAETP
LSPVNYWGQGTQVTVS

In some embodiments, the single domain antibodies disclosed herein is humanized. As used herein the term “humanized” refers to a single domain antibody of the invention wherein an amino acid sequence that corresponds to the amino acid sequence of a naturally occurring VHH domain has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional chain antibody from a human being. Methods for humanizing single domain antibodies are well known in the art. Typically, the humanizing substitutions should be chosen such that the resulting humanized single domain antibodies still retain the favourable properties of single domain antibodies of the invention. The one skilled in the art is able to determine and select suitable humanizing substitutions or suitable combinations of humanizing substitutions.

In some embodiment, the single domain antibody of the present invention is fused to a heterologous polypeptide to form fusion protein. As used herein, a “fusion protein” comprises all or part (typically biologically active) of a single domain antibody of the present invention operably linked to a heterologous polypeptide (i.e., a polypeptide other than the same single domain antibody). Within the fusion protein, the term “operably linked” is intended to indicate that the polypeptide of the invention and the heterologous polypeptide are fused in-frame to each other. The heterologous polypeptide can be fused to the N-terminus or C-terminus of the single domain antibody of the invention. In some embodiment, the heterologous polypeptide is fused to the C-terminal end of the single domain antibody of the present invention. In some embodiments, the heterologous polypeptide is a polypeptide that facilitates purification radiolabelling. In some embodiments, the single domain antibody of the present invention is fused to a polyhistidine tag (His-tag). The polyhistidine tag can enable the singled domain antibody to be purified then to be site-specifically labelled with a radionuclide complex.

The single domain antibody of the present invention is produced by any technique known in the art, such as, without limitation, any chemical, biological, genetic or enzymatic technique, either alone or in combination. For example, knowing the amino acid sequence of the desired sequence, one skilled in the art can readily produce said single domain antibody, by standard techniques for production of polypeptides. For instance, they can be synthesized using well-known solid phase method, preferably using a commercially available peptide synthesis apparatus (such as that made by Applied Bio systems, Foster City, Calif.) and following the manufacturer's instructions. Alternatively, the single domain antibody of the present invention can be synthesized by recombinant DNA techniques well-known in the art. For example, the single domain of the present invention can be obtained as DNA expression products after incorporation of DNA sequences encoding the single domain antibody into expression vectors and introduction of such vectors into suitable eukaryotic or prokaryotic hosts that will express the desired single domain antibody, from which they can be later isolated using well-known techniques. A variety of expression vector/host systems may be utilized to contain and express the single domain antibody of the present invention. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells.

As used herein, the term “radionuclide” has its general meaning in the art and refers to atoms with an unstable nucleus, characterized by excess energy available to be imparted either to a newly created radiation particle within the nucleus or via internal conversion. During this process, the radionuclide is said to undergo radioactive decay, resulting in the emission of gamma ray(s) and/or subatomic particles such as alpha or beta particles. These emissions constitute ionizing radiation. Radionuclides occur naturally, or can be produced artificially. Accordingly, the term “radiolabeled” refers to the radioisotopic labeling of the single domain antibody, wherein the said single domain antibody is labelled by including, coupling, or chemically linking a radionuclide to its amino acid sequence structure. Examples of suitable radionuclides which can be linked to the disclosed single domain antibody of the present invention can for example without any limitation be chosen from the group consisting of γ-emitting and α-emitting radioisotopes and β-emitting radioisotopes, including but not limited to a radioisotope chosen from the group consisting of Actinium-225, Astatine-211, Bismuth-212, Bismuth-213, Caesium-137, Chromium-51, Cobalt-60, Cupper-64 Dysprosium-165, Erbium-169, Fermium-255, Fluor-18, Gallium-67, Gallium-68, Gold-198, Holmium-166, Indium-111, Iodine-123, Iodine-124, Iodine-125, Iodine-131, Iridium-192, Iron-59, Lead-212, Lutetium-177, Molybdenum-99, Palladium-103, Phosphorus-32, Potassium-42, Rhenium-186, Rhenium-188, Samarium-153, Technetium-99m, Radium-223, Ruthenium-106, Sodium-24, Strontium-89, Terbium-149, Thorium-227, Xenon-133, Ytterbium-169, Ytterbium-177, Yttrium-90.

There are various radiolabeling strategies available to incorporate a radionuclide into a protein. The choice of technique for a radiochemist depends primarily on the radionuclide used. For example, the radioactive isotopes of iodine possess the ability to be directly integrated into a molecule by electrophilic substitution or indirectly via conjugation. Unlike many metallic radionuclides which possess the ability to form stable complexes with chelating agents, thus allowing for conjugation with a protein. Appropriate chelation ligands can be readily incorporated into the disclosed single domain antibody of this invention by the methods previously described for radionuclides. Such chelation ligands can include, but are not limited to, diethylenetriaminepentaacetic acid (DTPA), ethylenediaminetetraacetic acid (EDTA), 1,4,7-triazacyclononane-triacetic acid (NOTA), N,N-bis(2-hydroxybenzyl)ethylenediamine-N,N-diacetic acid (HBED), and tetraazacyclododecanetetraacetic acid (DOTA), and other macrocycles known to those skilled in the art. Alternatively the single domain antibody is admixed with a salt of the radioactive metal in the presence of a suitable reducing agent, if required, in aqueous media at temperatures from room temperature to reflux temperature, and the end-product coordination complex can be obtained and isolated in high yield at both macro (carrier added, e.g., Tc-99) concentrations and at tracer (no carrier added, e.g., Tc-99m) concentrations (typically less than 10⁻⁶ molar). Technetium-99m is the most commonly used radionuclide in diagnostic nuclear medicine. The Tc metal coordination complexes can be prepared by methods known in the art. It is well established that when [^{99m}Tc] pertechnetate ([^{99m}TcO₄]⁻, (31)) is reduced by a reducing agent, such as stannous chloride, in the presence of chelating ligands such as, but not restricted to, those containing N₂S₂, N₂SO, N₃S and NS₃ moieties, complexes of (TcO)N₂S₂, (TcO)N₂SO, (TcO)N₃S and (TcO)NS₃ are formed (Meegalla et al. J. Med. Chem., 40:9-17, 1997). Another preferred method for radio labeling the single domain antibody involves the use of glucoheptonate together with a reducing agent such as stannous chloride to label the chelation moiety on the single domain antibody (Lister-James, et al., J Nucl Med 37:775-781, 1997; Meegalla, et al., J Med Chem 40:9-17, 1997). Another preferred labeling method involves one-step labeling of His-tagged single domain antibody with Tc(I)-carbonyl complexes (Waibel, et al., Nature Biotechnology, 17:897-901, 1999). Such Tc-99m labeling and chelating moieties can be incorporated into potential receptor-selective imaging agents (Horn and Katzenellenbogen, Nucl. Med. Biol., 24:485-498, 1997). The incorporation of such moieties, specifically those that chelate radioactive metals or other metals of interest for imaging (e.g., magnetic resonance relaxivity metals) or radiotherapy, into other single domain antibody motif via the use of a functional linker, thereby enabling selective cellular delivery and retention of the metal coordination complex, is new. In some embodiments, the single domain antibody of the present invention is radiolabeled the using tricarbonyl method at a C-terminal Histine-tag (e.g. hexahistidine-tag). In fact, this incorporation of His-Tag can be used not only for immobilized metal affinity chromatography (IMAC) purification, but also in principle for site-specific labeling with ^{99m}Tc-tricarbonyl ([^{99m}Tc(CO₃(H₂O)₃]⁺) (Waibel, et al., Nature Biotechnology, 17:897-901, 1999) such as described in the EXAMPLE.

As a non-limiting example, the radiolabeled single domain antibody of the present invention may be of use in diagnosing or confirming the diagnosis of a cancer that expresses mesothelin in a subject. In some embodiments, the subject suffers from a cancer selected from the group consisting of mesothelioma, prostate cancer, lung cancer, stomach cancer, squamous cell carcinoma, pancreatic cancer, cholangiocarcinoma, breast cancer and ovarian cancer. In some embodiments, the radiolabeled single domain antibody of the present invention is particularly suitable for imaging cancer, and in particular imaging metastatic cancer. As used herein, the term “metastasis” has its general meaning in the art and refers to the spread of cancer beyond its originating site in the body. Thus, metastatic lesions are cancerous tumors that are found in locations apart from the original starting point of the primary tumor. Metastatic tumors occur when cells from the primary tumor break off and travel to distant parts of the body via the lymph system and blood stream. The term “metastatic cancer” as used herein refers to late-stage cancer and to the medical classification of cancer as being in stage III, when cancer cells are found in lymph nodes near the original tumor, or in stage IV, when cancer cells have traveled far beyond the primary tumor site to distant parts of the body. Metastatic lesions are most commonly found in the brain, lungs, liver, or bones. An individual with metastatic cancer might or might not experience any symptoms, and the symptoms could be related to the area where metastasized cells have relocated.

Accordingly, a further object of the present invention relates to a method of obtaining an image of a cancer in a subject in need thereof comprising i) administering to the subject a pharmaceutically acceptable composition comprising the radiolabeled single domain antibody of the present invention; ii) identifying a detectable signal from the radiolabeled single domain antibody in the subject and iii) generating an image of the detectable signal, thereby obtaining an image of the cancer in the subject.

In some embodiments, the signal is detected by Single-Photon Emission Computed Tomography (SPECT) or Positron Emission Tomography (PET). The term “SPECT” as used herein refers to “Single-Photon Emission Computed Tomography which is a nuclear medicine tomographic imaging technique using gamma rays. It is very similar to conventional nuclear medicine planar imaging using a gamma camera and able to provide true 3D information. This information is typically presented as cross-sectional slices through the patient, but can be freely reformatted or manipulated as required. In SPECT, gamma-emitting isotopes, herein referred to as radiopharmaceuticals, are injected into a patient. The basic technique requires delivery of a gamma-emitting radioisotope (called radionuclide) into the patient, normally through injection into the bloodstream. Preferred radionuclides for SPECT are Technetium-99m and Iodine-123. The term “Positron Emission Tomography (PET)” as used herein refers to a nuclear imaging technique used in the medical field to assist in the diagnosis of diseases. As SPECT PET allows the physician to examine the whole patient at once by producing pictures of many functions of the human body unobtainable by other imaging techniques. In this regard, as SPECT, PET displays images of how the body works (physiology or function) instead of simply how it looks. PET is considered the most sensitive, and exhibits the greatest quantification accuracy of any nuclear medicine imaging instrument available at the present time. In PET, positron-emitting isotopes, herein referred to as radiopharmaceuticals, are injected into a patient. Preferred radionuclides for PET are Fluor-18 and Gallium-68. When these radioactive drugs are administered to a patient, they distribute within the body according to the physiologic pathways associated with their stable counterparts. By way of example SPECT studies can be carried out using ^{99m}Tc and PET studies using ¹⁸F. The skilled person, however, will be aware of other suitable SPECT and PET radionuclides that can be employed in the present invention. The quantity of the radiolabeled single domain antibody should be an effective amount for the intended purpose. Such amounts can be determined empirically, and are also well known in the art. For example, amounts of the radiolabeled single domain antibody can be in the range of from about 37 MBq to about 3700 MBq mCi, more preferably from about 37 MBq to about 1850 MBq. This amount can be adjusted for body weight and the particular disease state, and can be about 1 37 MBq/kg body weight. Typically for SPECT performed with Tc-99m I-123 and In-111 the maximal dose ranges from 185 to 1110 MBq, 185 to 370 MBq, and 74 to 185 MBq respectively. Typically for PET performed with Ga-68, F-18 and Cu-64 the maximal dose ranges from 185 to 370 MBq, 185 to 370 MBq and 74 to 370 MBq respectively.

The radiolabeled single domain antibody of the present invention can also suitable for the treatment of cancer (i.e. radiotherapy).

Accordingly a further object of the present invention relates to a method of treating cancer in a patient in need thereof comprising administering to the subject a therapeutically effective amount of a radiolabeled single domain antibody of the present invention.

Detailed protocols for radiotherapy are readily available to the expert (Cancer Radiotherapy: Methods and Protocols (Methods in Molecular Medicine), Huddart R A Ed., Human Press 2002). The skilled person knows how to determine an appropriate dosing and application schedule, depending on the nature of the disease and the constitution of the patient. In particular, the skilled person knows how to assess dose-limiting toxicity (DLT) and how to determine the maximum tolerated dose (MTD) accordingly. Preferred radionuclide for alpha-therapy are ²¹¹At, 212Bi, ²¹³Bi, ²²³Ra and ²²⁵Ac. Preferred radionuclides for beta-therapy are Lutecium-177, and Yttrium-90. In some embodiments, the therapeutic dose is between about 300 MBq and about 20000 MBq, between about 400 MBq and about 20000 MBq, between about 500 MBq and about 20000 MBq, between about 1000 MBq and about 20000 MBq, between about 2000 MBq and about 20000 MBq, between about 3000 MBq and about 20000 MBq, between about 4000 MBq and about 20000 MBq, between about 5000 MBq and about 20000 MBq, between about 10000 MBq and about 20000 MBq, between about 5000 MBq and about 20000 MBq, between about 10000 MBq and about 20000 MBq, between about 300 MBq and about 10000 MBq, between about 400 MBq and about 10000 MBq, between about 500 MBq and about 10000 MBq, between about 1000 MBq and about 10000 MBq, between about 2000 MBq and about 10000 MBq, between about 3000 MBq and about 10000 MBq, between about 4000 MBq and about 10000 MBq, or between about 5000 MBq and about 10000 MBq. Typically for beta-radiotherapy performed with Lu-177, Y-90 and I-131, the maximal dose ranges from 1850 to 37000 MBq, 1850 to 37000 MBq and 370 to 37000 MBq respectively. Typically for alpha-radiotherapy performed with At-211 or Ac-225 the maximal dose is 0.1 MBq/kg. Of course, these amounts can be tailored to meet the specific requirements of the disease state being treated, and can also vary depending upon the weight and condition of the patient as is well known in the art. Note, for example, Clinical Nuclear Medicine, 1998, Third Edition, Chapman & Hall Medical. The regimen for treating a patient with the compounds and/or compositions of the present invention is selected in accordance with a variety of factors, including the age, weight, sex, diet, and medical condition of the patient, the severity of the condition, the route of administration, pharmacological considerations such as the activity, efficacy, pharmacokinetic, and toxicology profiles of the particular pharmacologically active compounds employed. Administration of the radiolabeled single domain antibody disclosed herein should generally be continued over a period of several days, weeks, months, or years. Patients undergoing treatment with the single domain antibody disclosed herein can be routinely monitored to determine the effectiveness of therapy for the particular disease or condition in question.

The radiolabelled single domain antibodies of the present invention can be administered by a variety of routes but parenteral administration is preferred, especially by intravenous, intramuscular, subcutaneous, intracutaneous, intraarticular, intrathecal, and intraperitoneal infusion or injection, including continuous infusions or intermittent infusions with pumps available to those skilled in the art. Alternatively, the radio labelled single domain antibodies can be administered by means of micro-encapsulated preparations, for example those based on liposomes as described in European Patent Application 0 213 523.

The radio labeled single domain antibodies of the present invention can be formulated as pharmaceutical compositions. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

The present invention also provides kits comprising the single domain antibody of the present invention and a radionuclide. Such kits can contain a predetermined quantity of single domain antibody and a predetermined quantity of a preselected radionuclide. The single domain antibody can be lyophilized to facilitate storage stability. The single domain antibody can be contained in a sealed, sterilized container. Instructions for carrying out the necessary reactions, as well as a reaction buffer solution(s), can also be included in the kit.

The invention will be further illustrated by the following figures and examples. However, these examples and figures should not be interpreted in any way as limiting the scope of the present invention.

### FIGURES

FIG. 1: Radio-HPLC profiles of ^{99m}Tc-A1 and ^{99m}Tc-C6 immediately after radiolabeling (A and B, respectively) and 2 hours post-injection to mice (C and D, respectively).

FIG. 2. In vivo biodistribution of ^{99m}Tc-A1 and ^{99m}Tc-C6 in HCC70 and MDA-MB-231 tumor xenografts. (A) Representative sagittal, coronal and transversal views of fused SPECT/CT images of HCC70 and MDA-MB-231 tumor-bearing mice at 1 h after i.v injection of ^{99m}Tc-A1 or ^{99m}Tc-C6. (B) In vivo quantification of ^{99m}Tc-A1 and ^{99m}Tc-C6 tumor uptake from SPECT images. (C) Ex vivo quantification of ^{99m}Tc-A1 and ^{99m}Tc-C6 tumor uptake from post-mortem biodistribution studies. Results were expressed as % ID/g of tumor. Statistics: ##p<0.01 vs MDA-MB-231 A1, ###p<0.001 vs MDA-MB-231 A1, **p<0.01 vs HCC70+Control Nanobody ***p<0.001 vs HCC70 Control Nanobody, ††p<0.01 vs HCC-70 A1. (B) In vivo quantification of 99mTc-A1 and 99mTc-C6 tumor uptake from SPECT images. (C) Ex vivo quantification of 99mTc-A1 and 99mTc-C6 tumor uptake from post-mortem biodistribution studies. Results were expressed as % ID/g of tumor. Statistics: ##p<0.01 vs MDA-MB-231 A1, ###p<0.001 vs MDA-MB-231 A1, **p<0.01 vs HCC70+Control Nanobody ***p<0.001 vs HCC70 Control Nanobody, ††p<0.01 vs HCC-70 A1.

FIG. 3: In vivo competition study. ^{99m}Tc-A1 was injected in HCC70-tumor bearing mice either alone (n=5), or together with a 150-fold excess of unlabeled A1 (n=5). (A) Representative SPECT/CT images of HCC70 tumor-bearing mice injected with ^{99m}Tc-A1 (left) or ^{99m}Tc-A1 and competitor (right), with sagittal view at the top and coronal view at the bottom. (B) Quantification of SPECT acquisitions. Results were expressed as % ID/g of tissue. **p<0.01 vs HCC70 A1+competition.

### EXAMPLE

Material & Methods

Ethic Statement and Mice

Four weeks-old female Balb/c athymic nude (BALB/c nu/nu) mice were purchased from Janvier Labs. All experiments were approved by the local ethic committee-ComEth-Grenoble Alpes University and the ad hoc French minister (APAFIS #3690-20 160 1 1916045217 v4).

Cell Lines and Culture Conditions

Two TNBC cell lines were used during the study. The HCC70 cell line was kindly provided by Dr. Molla A. (Institute for Advanced Biosciences, Université Grenoble Alpes, France) and was cultured using RPMI-1640 medium (PAN BIOTECH), supplemented with 2 mM L-Glutamine, 1 mM Sodium Pyruvate, 10 mM Hepes, 10% fetal bovine serum, and 1% Penicillin-Streptomycin. MDA-MB-231 cells were cultured with DMEM (PAN BIOTECH) supplemented with 2 mM L-glutamine, 1 mM Sodium Pyruvate, 10% fetal bovine serum, and 1% penicillin-streptomycin.

Western Blot Analysis

Mesothelin protein expression was determined by Western Blot analysis. HCC70 and MDA-MB-231 cells were cultured in 6-well plates during 48 h. Cells were washed with PBS and lysed using 200 μL of RIPA buffer [150 mM NaCl, 0.1% SDS, 0.5% Sodium Deoxycholate, Tris-HCl 50 mM (pH 8.0), sodium orthovanadate 1 nM, and protease inhibitor cocktail 1% (Sigma)]. Cell lysate was centrifuged at 10,000 g for 10 min at 4° C. and the supernatant was collected. Samples were assayed by BCA method (Pierce) and 30 μg of proteins were prepared for electrophoresis in a Laemmli sample buffer, containing β-mercaptoethanol. Samples were heated at 95° C. during 5 min and separated using a SDS-polyacrylamide gel (4/15%), and then transferred onto a nitrocellulose membrane. The membrane was incubated with the anti-mesothelin antibody (1/2000, Rabbit anti-MSLN Boster immunoleader) in PBS-Tween 0.1% BSA 1% overnight at 4° C., followed by the anti-rabbit IgG 1/2000 (Horseradish peroxidase-labeled goat anti-rabbit IgG; Dako) for 1 h at room temperature, after which the revelation was assessed using the chemiluminescence ECL kit (Biorad). As a loading control, the membrane was stripped and reprobed with an anti-β-actin antibody (BD).

Labeling Procedure

Materials

Tricarbonyl kit (Psi, Switzerland) contains the following lyophilized ingredients: 4.5 mg sodium boranocarbonate, 2.9 mg sodium tetraborate.10H₂O, 7.8 mg of sodium carbonate and 9 mg sodium tartrate.4H₂O. Acetonitrile, trifluoroacetic acid, 1-octanol with High Pressure Liquid Chromatography (HPLC) grade, were purchased from Aldrich Chemical Co. (St Louis, USA). Ultrapure water was product by a Milli-Q water purification system from Millipore (St-Quentin en Yvelines, France). A HPLC apparatus (Shimadzu) equipped with NaI (Tl) scintillation detectors (LabLogic, UK) was used for all analyses including labeling efficiency of radiolabeled products immediately and 6 h after labeling and also in vitro and in vivo blood stability studies. A symmetry C4 column from Waters (USA) was used. Activities of all samples were assessed using a dose calibrator Capintec CRC-15R (Aries, USA).

Circular Dichroism

Thermal stability of A1 and C6 was performed using Circular Dichroism (CD). The measurements were performed using a J715 spectropolarimeter (Jasco, Tokyo, Japan) in the far-UV of 205-260 nm region. Each sample with a concentration of 0.4 mg/ml in a total volume of 200 μL was placed in a cuvette with a 0.1 cm cell path length. Heat-induced unfolding was monitored by increasing the temperature from 25° C. to 80° C. The CD spectra were recorded at five points of 25, 50, 65, 75 and 80° C.

Radiolabeling with ^{99m}Tc

A1 and C6 were radiolabeled with technetium-99m (^{99m}Tc-A1 or ^{99m}Tc-C6) using tricarbonyl method at their C-terminal hexahistidine-tag (His-Tag). The radio labeling was performed in two steps. First 1 mL of freshly eluted ^{99m}TcO₄⁻ solution (1.5-3 GBq from a ⁹⁹Mo/^{99m}Tc generator, Drytec, GE healthcare Piscataway, N.J.) was added to a tricarbonyl kit and was then incubated at 100° C. for 20 min. After cooling to room temperature, the freshly prepared ^{99m}Tc(H₂O)₃(CO)₃]⁺ (^{99m}Tc-tricarbonyl) was neutralized with 1 M HCl to adjust the pH to 6-7. Second, 500 μL of Tc-tricarbonyl were added to a solution containing 50 μg of nanobody and incubated for 60 min at 60° C. The radiolabeled nanobodies were further purified by Sephadex G25 columns (NAP-5; GE Healthcare, Piscataway, N.J.) in PBS and filtered through a 0.22 μm Millex filter (Millipore, Bedford, Mass.).

The radiochemical purity (RCP) of ^{99m}Tc-A1 or ^{99m}Tc-C6 was determined immediately after labeling by radio-HPLC using a C4 column (Symmetry 300 C4, 3.5 μm, 4.6 mm×150 mm) with a gradient mobile phase of 0-5 min: 5% solvent B; 5-20 min: 5%-60% solvent B; 20-25 min: 60% solvent B; 25-30 min: 60%-5% solvent B at a flow rate of 1 mL/min. The 2 solvents were: solvent A with 0.1% TFA in water (v/v) and solvent B with 0.1% TFA, 90% ACN (v/v). Radioactivity was monitored using a radiodetector (γ-RAM Model 4, LabLogic). The RCP of both radio labeled nanobodies was also assessed 6 h following labeling using the same protocol.

Lipophilicity

The lipophilicity of ^{99m}Tc-A1 or ^{99m}Tc-C6 was evaluated using an octanol-phosphate-buffered saline (PBS) distribution study. The radiolabeled nanobody (20-30 μL, ˜11 MBq) was added to 1 mL of 1:1 n-octanol/PBS mixture. After mixing for 1 min, the solution was centrifuged for 3 min at 13,000 rpm to ensure complete separation of layers. The activity of each layer was measured separately using a dose calibrator (Capintec CRC-15R). This process was repeated by replacing fresh phosphate buffer and 1-octanol, respectively. The partition coefficient (log P) was calculated as the radioactivity ratio of the organic phase to aqueous phase using the following formula: Log P=log (total counts in 1-octanol)/(total counts in PBS buffer). Log P values for each compound were determined in triplicate.

In Vitro Blood Distribution Pattern Stability in Human Blood

The in vitro stability of both ^{99m}Tc-nanobodies was evaluated in human blood. 74 MBq of radiolabeled compound (^{99m}Tc-A1 or ^{99m}Tc-C6) was added to 1 mL human blood and incubated at 37° C. A sample of 120-150 μL of whole blood was immediately withdrawn before incubation at 37° C. corresponding to the 0 min time point (radiotracer in contact with human blood without incubation). After 0.5 h, 1 h, 2 h, 4 h and 6 h, a whole blood sample was removed and then centrifuged (7,700 rpm, 2 min) to separate the plasma from blood cells. The activities bound to blood cells and in the plasma were measured in the pellet and supernatant, respectively, using a dose calibrator. Tricholoroacetic acid (TCA 10%, 5 μL) was added to the plasma fraction. The sample was then centrifuged at 13,000 rpm for 3 min to separate the plasma proteins in the pellet from the protein-free plasma in the supernatant. The activity was measured in each fraction. The results were expressed as percent of total blood tracer activity contained in blood cells, plasma proteins, and protein-free plasma fractions. To determine the in vitro stability of each radiolabeled nanobody, the fraction corresponding to the protein-free plasma was analyzed with radio-HPLC using conditions similar to those described above. The experiments were performed in triplicate.

In Vivo Stability in Mouse Blood

The evaluation of the in vivo stability of^{99m}Tc-nanobodies (A1 or C6) in mouse blood was performed as follows: 2 hours following intravenous injection, the animals were anesthetized (isoflurane) and a transmural puncture was performed in order to perform blood withdrawal directly from the left ventricular cavity. The blood sample was immediately centrifuged and plasma proteins were then precipitated using TCA 10% and further centrifuged at 13,000 rpm for 3 min. The protein-free plasma fraction was analyzed by radio-HPLC as described above.

Saturation Binding Experiments with ^{99m}Tc-A1 and ^{99m}Tc-C6 on Recombinant MSLN

Human MSLN recombinant protein (100 ng, R&D systems) was immobilized on immunosorbent plates (Corning Costar Stripwell, Sigma Aldrich) overnight at 4° C., and blocked with 1% milk. Serial dilutions of ^{99m}Tc-A1 and ^{99m}Tc-C6 from 1 μM to 0.8 nM were incubated for 1 hour. Unbound activity was removed by 5 serial washes with PBS-Tween 0.1%. The radioactivity in each well was determined using a γ-counter (Wizard², Perkin Elmer). Unspecific binding was determined by incubation of ^{99m}TC-A1 or ^{99m}Tc-C6 in empty wells. Results were corrected from background and decay, and the [^{99m}Tc-A1] and [^{99m}Tc-C6] binding curves were fitted using a nonlinear regression equation (specific binding: Y=B max*X/(K_{D}+X) with X being the radioligand concentration) and using GraphPad Prism 6 software to determine K_{D} values. Uptake was normalized to the Bmax for graphic representation of 3 independent experiments.

Saturation Binding with ^{99m}Tc-A1 and ^{99m}Tc-C6 on Cells

HCC70 and MDA-MB-231 (15.10⁴ cells) were grown for 48 hours in 96-well plates and then fixed in formalin. ^{99m}Tc-A1 and ^{99m}Tc-C6 (0.8-500 nM in PBS) were incubated for 1 hour at room temperature. The wells were then washed 5 times with PBS-Tween 0.1%. Radioactivity was determined using a γ-counter (Wizard², Perkin Elmer). Specific binding was calculated by subtracting non-specific binding on MSLN-negative MDA-MB-231 cells from total binding on MSLN-positive HCC70 cells. A nonlinear regression equation fit was performed (GraphPad Prism) and K_{D} values were determined (Specific binding: Y=B max*X/(K_{D}+X)).

In Vitro Competition Studies

HCC70 and MDA-MB-231 cells were rinsed with PBS, detached using EDTA 5 mM, and resuspended at 200,000 cells per tube. Cells were incubated for 1 hour with PBS-BSA 1% and then with 40 nM of ^{99m}Tc-A1 or 150 nM of ^{99m}Tc-C6 for 1 h at 4° C., in the absence or presence of a 200-fold excess of unlabeled A1 or C6. Cell suspensions were centrifuged at 400 g during 5 min at 4° C., and washed 5-times with cold-PBS. Cells were then transferred to new tubes and radioactivity was determined using a γ-counter (Wizard², Perkin). Results were expressed as fold/control with MDA-MB-231 as the control.

Tumor Model

To evaluate ^{99m}Tc-A1 and ^{99m}Tc-C6 biodistribution and tumor uptake, female BALB/c nu/nu mice (5 weeks old) were subcutaneously inoculated into the left flank with either HCC70 (3.5×10⁶, n=32) or MDA-MB-231 (2×10⁶, n=6) cells, in a 2:1 mixture of PBS and Matrigel (Corning®). The tumors were allowed to grow for 3-4 weeks to reach ˜400 mm³.

SPECT/CT Imaging

Mice were subdivided in 4 groups: HCC70 tumor-bearing mice injected with 1) ^{99m}Tc-A1 (HCC70-A1, n=8), 2) ^{99m}Tc-C6 (HCC70-C6, n=7) or 3) irrelevant ^{99m}Tc-CTL (HCC70-CtTL, n=7), and 4) MDA-MB-231 tumor-bearing mice injected with ^{99m}Tc-A1 (MDA-MB-231, n=6). SPECT/CT acquisitions were performed 1 hour after intravenous injection of 49.1±13.7 MBq of ^{99m}Tc-A1, ^{99m}Tc-C6 or ^{99m}Tc-CTL. Mice were anesthetized using 2% isoflurane in a 1:1 mixture of room air and oxygen and then were placed in a bed for whole body SPECT/CT acquisitions (nanoSPECT; Bioscan/Mediso). First, a CT acquisition was performed during 8 minutes using the following acquisition parameters: 45 kVp, 240 projections and 500 ms/projections. Then, the SPECT acquisition was performed with 4 heads equipped with multipinhole collimators using 24 projections and 45 min of acquisition. CT and SPECT acquisitions were reconstructed and fused using InVivoScope software (inviCRO). For competition studies, HCC70 tumor-bearing mice were injected with 17.6±5.3 MBq of ^{99m}Tc-A1 (n=5) with or without a 150-fold excess of unlabeled A1 nanobody (n=5). SPECT/CT were performed as described above.

SPECT/CT Quantification

SPECT quantification was performed on the basis of the CT data. A sphere of 50 mm³ was drawn at the center of the tumor on CT image. ^{99m}Tc-nanobody activity was expressed in % ID/cm³.

Ex-Vivo Biodistribution of ^{99m}Tc-A1

Two hours after injection and immediately following SPECT/CT image acquisition, anesthetized mice were euthanized using CO₂, and tumors were harvested along with others organs. Tissues were weighed ant tracer activity was determined with a y-counter (Wizard², Perkin). Results were corrected for decay, injected dose (ID) and weight and expressed as % ID/g. Tumor-to-muscle and tumor-to-blood activity ratios were computed.

Immunohistochemistry

HCC70 and MDA-MB-231 tumors were fixed using acetone during 10 minutes at −20° C. and 10-μm thick cryosections were obtained. Immunohistochemistry was performed using mesothelin staining with A1 nanobody (20 μg/mL) or commercial antibody (polyclonal anti-mesothelin 0.5 μg/mL, Boster immunoleader), using DAB as the chromogen (Vector).

Statistics

Data were expressed as mean±standard deviation (SD) and compared using an unpaired Mann-Whitney test for inter group analysis. P values<0.05 were considered significant. Significance of linear correlation was assessed with Pearson's test. The significance level was set at p<0.05.

Results

Circular Dichroism

During the radiolabeling procedure, a heating step is necessary. Potential thermal unfolding of A1 or C6 was monitored by Circular Dichroism (CD) to determine the maximal temperature at which the radiolabeling step could be performed without affecting the secondary structure of the proteins.

No shift was detected up to 65° C. (data not shown). Thermal unfolding was observed at 75° C. and 80° C. The maximal temperature used for the radiolabeling procedure was therefore set at 65° C.

Radiolabeling with ^{99m}Tc and Lipophilicity

A1 and C6 were successfully radiolabeled with technetium-99m using the tricarbonyl method. Radiochemical purity of radiolabeled products was higher than 99% immediately after radiolabeling and purification steps for both nanobodies. Moreover, ^{99m}Tc-A1 and ^{99m}Tc-C6 remained stable for 6 hours after labeling (data not shown). The lipophilicity of both radiolabeled nanobodies was determined by 1-octanol/PBS partition coefficient. The Log P values were −1.8±0.5 for ^{99m}Tc-A1 and −2.3±0.8 for ^{99m}Tc-C6.

MSLN Expression in Human Breast Cancer Cells

Western blot analysis was performed to determine the expression of the MSLN protein in the TNBC cell lines HCC70 and MDA-MB-231. HCC70 cells expressed MSLN protein, whereas MDA-MB-231 cells did not (data not shown).

^{99m}Tc-A1 and ^{99m}Tc-C6 Affinity for MSLN

^{99m}Tc-A1 affinity was more than 2-fold higher than ⁹⁹ m Tc-C6 affinity (K_{D}=43.9±4.0 nM and 107.3±15.9 nM respectively, data not shown). Similar results were obtained using an ELISA assay performed on MSLN-expressing HCC70 cells (data not shown).

In Vitro Competition

For the competition study ^{99m}Tc-A1 and ^{99m}Tc-C6 were incubated with MDA-MB-231 or HCC70 at their respective K_{D} in the presence or absence of a 200-fold excess of unlabeled nanobody. ^{99m}Tc-A1 and ^{99m}Tc-C6 binding to MSLN-positive HCC70 cells was respectively 7.9- and 4.6-fold higher than on MSLN-negative MDA-MB-231 cells (data not shown). Moreover, the competition resulted in a significant 6-fold and 3.5-fold decrease in ^{99m}Tc-A1 and ^{99m}Tc-C6 binding to HCC70 cells (p<0.001) (data not shown).

Blood Distribution Patterns and In Vitro Stability in Human Blood

The results indicated that only 14-20% of ^{99m}Tc-A1 and 10-18% of ^{99m}Tc-C6 were associated with blood cells and plasma proteins (data not shown). Both nanobodies were therefore primarily associated with the protein-free plasma fraction (63-75%).

The protein-free plasma fraction was then analyzed by HPLC to determine the in vitro stability of radiolabeled nanobodies. A good stability was observed for both nanobodies with a RCP higher than 98% (Table 1).

In Vivo Stability in Mouse Blood

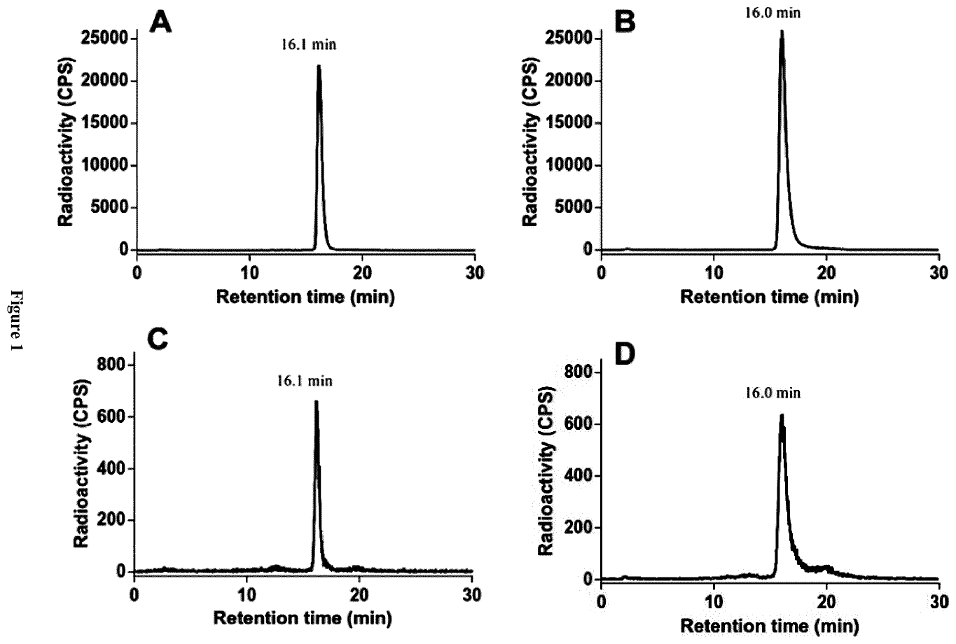

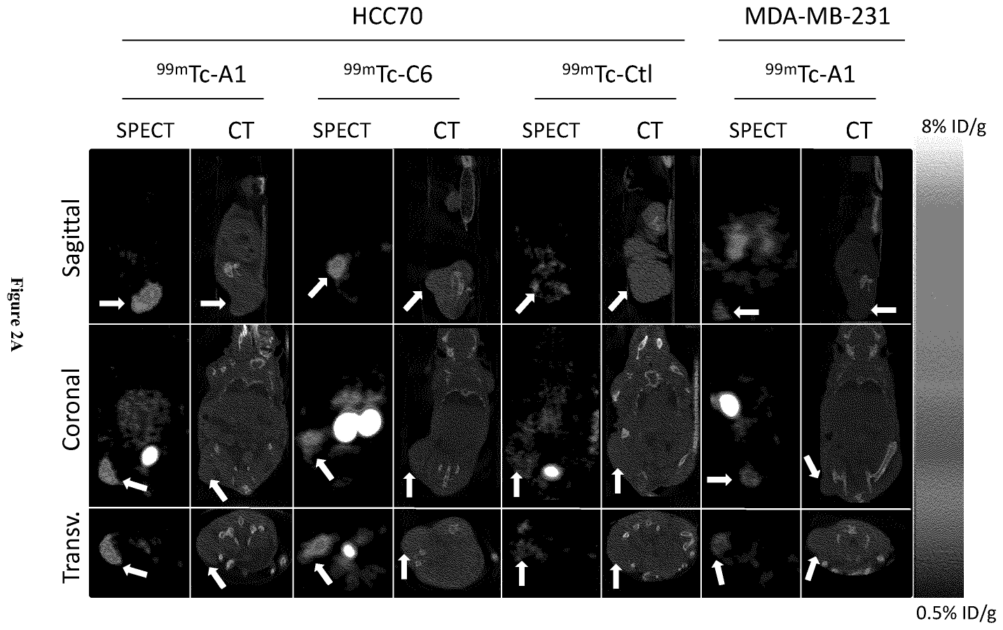

A single major peak corresponding to the nanobody was observed for both tracers. Absence of alternative radioactive products on the profiles confirmed the in vivo stability of ^{99m}Tc-A1 and ^{99m}Tc-C6 (FIG. 1).

In Vivo SPECT/CT Imaging of Tumor-Bearing Mice

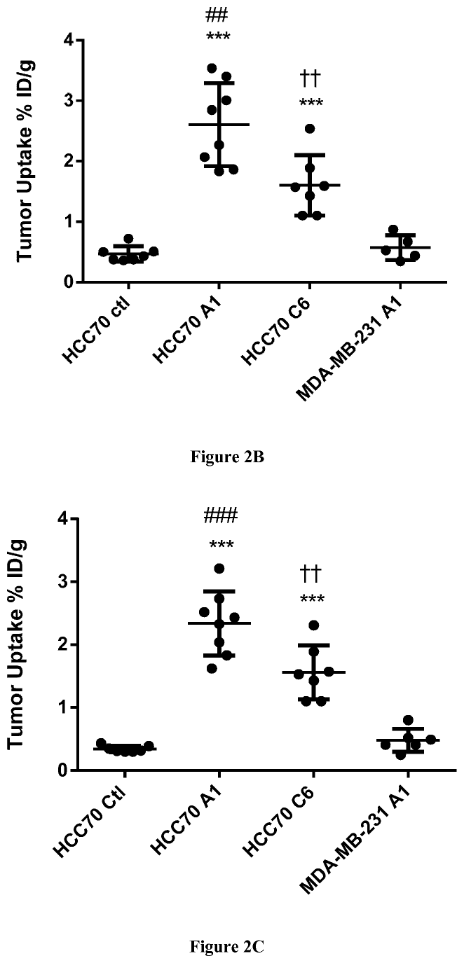

FIG. 2A shows sagittal, coronal and transversal views of fused SPECT/CT images. ^{99m}Tc-A1 and ^{99m}Tc-C6 uptake in MSLN-positive HCC70 tumors was readily identifiable, whereas a weak signal was observed with the irrelevant control nanobody in HCC70 tumor, or with ^{99m}Tc-A1 in MSLN-negative MDA-MB-231 tumor. Interestingly, ^{99m}Tc-C6 uptake in HCC70 tumors was visually lower than that of ^{99m}Tc-A1. Furthermore, liver accumulation was observed for ^{99m}Tc-C6 but not for ^{99m}Tc-A1. Nonspecific kidney elimination was observed in all groups.

Those observations were confirmed by image quantification showing that ^{99m}Tc-A1 activity was 5-fold higher than ^{99m}Tc-CTL activity in HCC70 tumor-bearing mice (2.6±0.7 vs 0.5±0.13% ID/g, p<0.01). In addition, ^{99m}Tc-A1 uptake in HCC70 tumor was 4-fold higher than that observed in MDA-MB-231 tumors (2.6±0.7 vs 0.6±0.2% ID/g, p<0.001) (FIG. 2B). HCC70 ^{99m}Tc-C6 uptake was also significantly higher than that of ^{99m}Tc-CTL (p<0.001) but remained ˜2-fold lower than that of ^{99m}Tc-A1 (1.4±0.3 vs 2.6±0.7% DI/g, respectively, p<0.01). Ex vivo quantification of tracer activity by γ-well counting indicated similar results (FIG. 2C). Indeed, ^{99m}Tc-A1 uptake by HCC70 tumors was 6-fold higher than that of ^{99m}Tc-CTL and 5-fold higher than that observed for the same tracer on MSLN-negative MDA-MB-231 tumors (2.3±0.4 vs 0.3±0.1 and 0.5±0.2% DI/g respectively, p<0.001 for both comparisons). Moreover, ^{99m}Tc-C6 uptake was significantly lower than that of ^{99m}Tc-A1 in HCC70 tumors (1.6±0.4 vs 2.3±0.4% DI/g, p<0.01). In vivo absolute quantification of anti-MSLN nanobodies tumoral uptake from SPECT images was therefore accurate considering its significant correlation with the ex vivo-determined biodistribution data (Y=1.08×+0.09, r²=0.97, p<0.001).

Biodistribution of ^{99m}Tc-A1 and ^{99m}Tc-C6 in HCC70 and MDA-MB-231 Tumor-Bearing Mice

The results from 2 hrs-biodistribution studies following ^{99m}Tc-A1, ^{99m}Tc-C6 or ^{99m}Tc-CTL intravenous injections are summarized in Table 2. Significant, >200% ID/g kidney activity was observed for all nanobodies and in all groups. ^{99m}Tc-A1 uptake was <1% ID/g for all investigated organs with the exceptions of the kidney and tumors. In comparison, ^{99m}Tc-C6 uptake was >1% ID/g in the liver and significantly higher than that of ^{99m}Tc-A1 and ^{99m}Tc-Ctle in the stomach, liver and intestine (p<0.01). The blood activity of all three nanobodies was <0.5% ID/g. Tumor-to-blood (T/B), and tumor-to-muscle (T/M) ratios were determined for each group. HCC70 A1 T/B ratio was 10-fold higher than that of the CTL group (10.3±4.4 vs 1.1±0.7, p<0.001). Similarly, the HCC70 T/M ratio was 5-fold higher in the A1 group in comparison to the CTL group (22.5±3.4 vs 4.0±1.8, p<0.001). ^{99m}Tc-C6 T/M and T/B ratios were also found to be increased with respect to the CTL group (p<0.01) but remained significantly lower than that of ^{99m}Tc-A1 (p<0.05 for both ratios).

In Vivo Competition

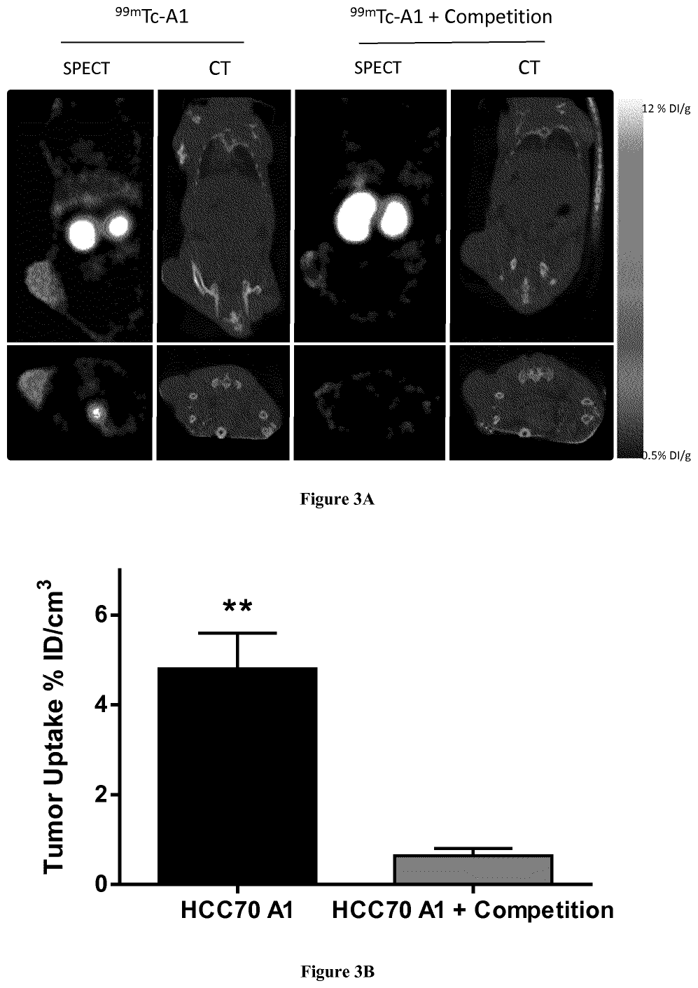

The in vivo co-injection of a 150-fold excess of unlabeled A1 induced a ˜7-fold decrease in ^{99m}Tc-A1 uptake in HCC70 tumors as determined by SPECT quantification (0.6±0.2% ID/g for HCC70 A1+competition vs 4.8±0.8% ID/g for HCC70 A1 alone, p<0.01) (FIG. 3).

Ex-vivo biodistribution confirmed this result with a ˜7-fold decrease in ^{99m}Tc-A1 uptake in HCC70 tumors (0.5±0.1% ID/g in comparison to 4.2±0.8% ID without competitor, p<0.01) (Table 3). With the exception of the kidneys, ^{99m}Tc-A1 uptake in all other investigated organs did not significantly changed. Consequently, a significant decrease was observed in T/B and T/M ratios (p<0.01 for both).

Anti-MSLN Immunohistochemistry on Tumor Xenografts

Mesothelin expression was evaluated by IHC on HCC70 and MDA-MB-231 xenografts using a commercially available antibody. As observed from cell culture experiments, HCC70 tumor xenograft expressed mesothelin, whereas MDA-MB-231 did not. Those results were further confirmed by IHC using the A1 nanobody as well as by Western blot analysis.

Discussion:

Most TNBC have an aggressive clinical course characterized by a high recurrence rate, more distant metastasis, and an overall decrease in survival in comparison with others forms of breast cancers [1]. TNBC are treated with chemotherapy or radiation therapy. However some TNBC are chemotherapy-resistant and researchers are still looking for the best combination of chemotherapeutics agents and other therapies such as immunotherapies. TNBC-antigens have recently been discovered and immunotherapies are under investigation, such as Trop2 targeting Antibody-drug-conjugate (IMMU-132) [26], or PD-L1 inhibitors [27].

Other potential targets have been identified and include MSLN [2, 3]. MSLN is a 40 kDa membrane-glycoprotein GPI-anchored which tissue expression is very limited (pericardium, pleura and peritoneum) and which is frequently overexpressed in most aggressive cancers such as pancreatic adenocarcinoma, ovarian cancers mesothelioma and TNBC. More specifically, MSLN is overexpressed in 10 to 20% of TNBC in association with (1) a high rate of metastasis, (2) a high recurrence rate, and (3) a decreased overall survival [18]. A number of therapies targeting MSLN-expressing tumors have been developed and are currently under clinical translation. Accordingly, SS1P is a recombinant immunotoxin consisting in an anti-mesothelin Fv of mice linked to Pseudomonas exotoxin A [28,29]. Preclinical studies using this compound showed complete remission of mesothelin-expressing tumor xenografts in mice [30]. The combination of SS1P with gemcitabine or Taxol® resulted in a marked anti-tumoral response [31,32]. Results from a Phase 1 clinical study showed significant anti-tumoral activity of SS1P in combination with chemotherapy in patients with unresectable, advanced pleural mesothelioma [33]. Based on studies showing that MSLN could elicit CD8+ T cell response in patients, tumor vaccines are under clinical investigations and have shown promising preclinical results [34,35]. Identification of patients with MSLN-expressing tumors can be performed by biopsy and blood testing with the Serum Mesothelin Related Peptide (SMRP) [24]. However, a discrepancy in the expression of tumor markers is often observed between the primary tumor and the metastasis that are not always accessible to biopsy. In addition, Concerning the SMRP blood test, if its level is increased in mesothelioma, it is not the case for pancreatic cancer despite an overexpression of the MSLN-membrane form in these tumors [36]. On the other hand, molecular nuclear imaging is well suited to determine a tumor's phenotype. mAbs radiolabeled with ⁶⁴Cu or ⁸⁹Zr have been evaluated and allowed the detection of MSLN expressing tumors in a xenograft pancreatic tumor model [37,38]. Nevertheless, the hepatic elimination and slow blood clearance of radiolabeled mAbs represented major limitations. Nanobody-based imaging agents characterized by a small size associated with fast blood clearance allow specific image acquisition with high target-to-background ratios as early as one hour following administration. Two MSLN-targeting nanobodies, A1 and C6 have been characterized by Prantner et al. Both exhibited high in vitro specificity and affinity for MSLN [25].

The objective of the present study was to perform the nuclear imaging of TNBC xenografts with ^{99m}Tc-labeled A1 and C6. (^{99m}Tc-A1 and ^{99m}Tc-C6). HCC70 were found to be MSLN-positive whereas MSLN-negative MDA-MB-231 was used as a control. ^{99m}Tc-A1 exhibited a high affinity for both MSLN (K_{D}=35 nM) as demonstrated in vitro on recombinant MSLN human protein and MSLN-expressing HCC70 cells. In vitro competition experiments on HCC70 cells confirmed the specificity of ^{99m}Tc-A1 binding. ^{99m}Tc-C6 affinity for human recombinant MSLN was 3-fold lower than that of ^{99m}Tc-A1. Those results were in accordance with that obtained by Prantner et al. using non-radiolabeled compounds, thereby indicating that the radio labeling method is suitable and that ^{99m}Tc-A1 and ^{99m}Tc-C6 can be further employed for in vivo evaluations. Both ^{99m}Tc-A1 and ^{99m}Tc-C6 remained stable over time in vitro and in vivo in vitro following incubation with human blood and in vivo intravenous administration to mice. Moreover, most of ^{99m}Tc-A1 and ^{99m}Tc-C6 remained in the protein-free plasma fraction thereby allowing good in vivo bioavailability. ^{99m}Tc-A1 and ^{99m}Tc-C6 enabled the non-invasive visualization of MSLN-positive tumors by SPECT imaging. High accumulation of ^{99m}Tc-A1 and ^{99m}Tc-C6 were observed in MSLN-positive HCC70 tumors whereas no signal was found in MSLN-negative MDA-MB-231 tumors. Moreover, ^{99m}Tc-A1 signal in HCC70 tumor was higher than that of ^{99m}Tc-C6. SPECT imaging quantification further confirmed those results with a 5-fold higher ^{99m}Tc-A1 uptake in HCC70 tumors than in MDA-MB-231 tumors. Moreover, the in vivo competition study demonstrated the specificity of ^{99m}Tc-A1 binding to MSLN. Renal accumulation was observed with both tracers, in accordance with general nanobody biodistribution features [39]. As a matter of fact, most nanobodies are exclusively eliminated through the kidneys and reuptake by the megalin-cubulin complex is responsible for their retention in the kidney complex. In addition to the tumor, competition studies also revealed a decrease of ^{99m}Tc-A1 retention in kidney, which could be explain by the saturation of megalin-cubulin complex by the competitor. Such saturation of the megalin-cubulin complex has been performed by other groups using gelofusin, a plasma substitute, resulting in a significant 40-50% decrease in kidney retention of the evaluated nanobodies.No signal was observed on in vivo SPECT images following ^{99m}Tc-A1 injection with the exception of the tumor, kidney and bladder, most likely as a result of the fact that (1) MSLN expression was very weak and limited to the pericardium, pleura and peritoneum, and (2) A1 was selected for its affinity for human MSLN. Interestingly, mild intensity signals were also observed in the stomach, liver and intestine following ^{99m}Tc-C6 injection, suggesting the involvement of the liver for 99mTc-C6 elimination.

Since 20% of women with breast cancer will develop distant metastasis within 5-years of diagnosis [40], an ideal imaging agent of TNBC should demonstrate high target-to-background ratio not only at the primary tumor site but also in the lungs, liver and bones, which are the most frequent metastatic sites of this type of cancer. Importantly, in the present study, only minimal uptake was observed in those organs with ^{99m}Tc-A1, but not ^{99m}Tc-C6. Taken together with its higher affinity and tumor uptake, this result suggests that ^{99m}Tc-A1 would be better suited for metastasis imaging.

Conclusion:

Due to the potential role of MSLN on the metastatic processes, identifying MSLN-expressing metastasis would allow to select patients that would benefit for anti-MSLN therapies. Two anti-MSLN nanobodies were tested for their ability to detect MSLN expressing tumor in vivo. The present in vitro and in vivo studies suggest that ^{99m}Tc-A1 is a promising tracer for MSLN-expressing tumor detection. ^{99m}Tc-A1 was therefore selected for further development, which might include the modification of, the original nanobody using DOTA chelation chemistry -in order to allow either ⁶⁸Ga or ¹⁷⁷Lu radio labeling for diagnosis or therapy as well as additional chemical engineering aimed at reducing renal uptake[41].

Tables:

TABLE 1
In vitro stability of ^{99m}Tc-A1 and ^{99m}Tc-C6 in human blood. The zero time point
refers to ratios observed immediately after contact with human blood. Results are
expressed as % of intact radiolabeled nanobody in protein-free plasma fraction

| Time | 0 h | 0.5 h | 1 h | 2 h | 4 h | 6 h |
|---|---|---|---|---|---|---|
| 99mTc-A1 | 99.8 ± 0.1 | 99.8 ± 0.1 | 99.7 ± 0.1 | 99.2 ± 0.6 | 98.7 ± 0.3 | 98.8 ± 0.5 |
| 99mTc-C6 | 99.6 ± 0.1 | 99.8 ± 0.1 | 99.2 ± 0.5 | 98.4 ± 0.4 | 98.2 ± 0.3 | 98.0 ± 0.2 |

TABLE 2
Ex-vivo Biodistribution of 99mTc-A1 and 99mTc-C6 in athymic nude mice bearing HCC70 and
MDA-MB-231 xenografts. Mice were euthanized 2 hours after intravenous injection of 99mTc-
A1, 99mTc-C6 or 99mTc-CTL. The organs were collected and weighed and radioactivity was
measured by γ-counter. Tumor-to-blood ratio and tumor-to-muscle ratio of 99mTc-A1
or 99mTc-Ctl were determined 2 hours post i.v injection. Results were expressed as %
ID/g. Mean and standard deviation have been corrected for radioactive decay of 99mTc.

| | HCC70 Ctle | HCC70 A1 | HCC70 C6 | MDA-MB-231 A1 |
|---|---|---|---|---|
| Target | (n = 7) | (n = 8) | (n = 7) | (n = 6) |
| Brain | 0.01 ± 0.01 | 0.01 ± 0.00 | 0.01 ± 0.00 | 0.01 ± 0.00 |
| Stomach | 0.35 ± 0.14 | 0.42 ± 0.06 | 0.67 ± 0.24**^{††} | 0.43 ± 0.07 |
| Intestine | 0.29 ± 0.10 | 0.36 ± 0.08 | 0.58 ± 0.30**^{††} | 0.30 ± 0.08 |
| Liver | 0.66 ± 0.15 | 0.74 ± 0.18^{#} | 1.25 ± 0.19**^{††} | 0.58 ± 0.06 |
| Pancreas | 0.16 ± 0.05 | 0.27 ± 0.07**^{#} | 0.22 ± 0.03 | 0.18 ± 0.03 |
| Heart | 0.24 ± 0.11 | 0.23 ± 0.04^{##} | 0.20 ± 0.04 | 0.15 ± 0.05 |
| Lungs | 0.68 ± 0.24 | 0.61 ± 0.10 | 0.80 ± 0.11 | 0.56 ± 0.27 |
| Kidney | 264.56 ± 31.77 | 268.81 ± 16.49^{##} | 255.21 ± 41.80 | 233.21 ± 13.14 |
| Skin | 0.42 ± 0.16 | 0.58 ± 0.13 | 0.44 ± 0.10^{†} | 0.49 ± 0.14 |
| Bones | 0.22 ± 0.07 | 0.22 ± 0.02^{#} | 0.21 ± 0.04 | 0.16 ± 0.01 |
| Ovaries | 0.42 ± 0.19 | 0.50 ± 0.14 | 0.61 ± 0.09 | 0.44 ± 0.18 |
| Lymph Nodes | 0.50 ± 0.35 | 0.44 ± 0.16 | 0.42 ± 0.04 | 0.33 ± 0.06 |
| Blood | 0.42 ± 0.24 | 0.25 ± 0.10 | 0.28 ± 0.07 | 0.19 ± 0.10 |
| Skeletal Muscle | 0.10 ± 0.04 | 0.10 ± 0.02 | 0.09 ± 0.02 | 0.09 ± 0.02 |
| Tumor | 0.34 ± 0.05 | 2.34 ± 0.36***^{###} | 1.56 ± 0.43***^{††} | 0.48 ± 0.18 |
| Tumor/Blood | 1.05 ± 0.69 | 10.32 ± 4.43***^{##} | 5.66 ± 1.12**^{†} | 3.09 ± 2.39 |
| Tumor/Muscle | 3.99 ± 1.78 | 22.46 ± 3.43***^{###} | 17.18 ± 3.60**^{†} | 5.26 ± 1.40 |

*p < 0.05,
**p < 0.01,
***p < 0.001 vs HCC70-Ctl.
^{#}p < 0.05,
^{##}p < 0.01 and
^{###}p < 0.001 vs MDA-MB-231 A1.
^{†}p < 0.05,
^{††}p < 0.01 vs HCC70-A1.

TABLE 3
Ex-vivo Biodistribution of 99mTc-A1 in athymic nude mice bearing
HCC70 xenografts. Mice were euthanized 2 hours after intravenous
injection of 99mTc-A1 in the presence or absence of an A1
excess. The organs were collected and weighed and radioactivity
was measured by γ-counter. Tumor-to-blood and tumor-to-
muscle ratios were also determined. The data were corrected
for background and decay and expressed as % ID/g.

| | HCC70 A1 | HCC70 A1 ± Competition |
|---|---|---|
| Target | (n = 5) | (n = 5) |
| Brain | 0..01 ± 0.00 | 0.01 ± 0.00 |
| Stomach | 0.59 ± 0.15 | 0.49 ± 0.23 |
| Intestine | 0.52 ± 0.30 | 0.42 ± 0.28 |
| Liver | 0.95 ± 0.24 | 0.72 ± 0.33 |
| Pancreas | 0.28 ± 0.03 | 0.19 ± 0.06 |
| Heart | 0.21 ± 0.08 | 0.22 ± 0.08 |
| Lungs | 0.79 ± 0.25 | 0.65 ± 0.29 |
| Kidney | 270.12 ± 15.70* | 180.76 ± 28.69 |
| Skin | 0.81 ± 0.34 | 0.86 ± 0.19 |
| Bones | 0.23 ± 0.07 | 0.28 ± 0.15 |
| Ovaries | 0.67 ± 0.19 | 0.50 ± 0.13 |
| Lymph Nodes | 0.63 ± 0.11 | 0.56 ± 0.13 |
| Blood | 0.30 ± 0.13 | 0.25 ± 0.14 |
| Skeletal Muscle | 0.14 ± 0.04 | 0.15 ± 0.05 |
| Tumor | 4.23 ± 0.83** | 0.54 ± 0.10 |
| Tumor/Blood | 14.49 ± 6.43** | 3.01 ± 1.66 |
| Tumor/Muscle | 35.26 ± 11.73** | 3.78 ± 1.20 |

*p < 0.05,
**p < 0.01 vs HCC70 + competition.

### REFERENCES

Throughout this application, various references describe the state of the art to which this invention pertains. The disclosures of these references are hereby incorporated by reference into the present disclosure.

1. Dent R, Trudeau M, Pritchard K I, et al. Triple-Negative Breast Cancer: Clinical Features and Patterns of Recurrence. Clin Cancer Res. 2007; 13(15):4429-4434. doi:10.1158/1078-0432.CCR-06-3045.

2. Crown J, O′Shaughnessy J, Gullo G. Emerging targeted therapies in triple-negative breast cancer. Ann Oncol. 2012; 23(suppl 6):vi56-vi65. doi:10.1093/annonc/mds196.

3. Andre F, Zielinski C C. Optimal strategies for the treatment of metastatic triple-negative breast cancer with currently approved agents. Ann Oncol. 2012; 23(suppl 6):vi46-vi51. doi:10.1093/annonc/mds195.

4. Chang K, Pastan I. Molecular cloning of mesothelin, a differentiation antigen present on mesothelium, mesotheliomas, and ovarian cancers. Proc Natl Acad Sci. 1996; 93(1):136-140.

5. Bera T K, Pastan I. Mesothelin is not required for normal mouse development or reproduction. Mol Cell Biol. 2000; 20(8):2902-2906.

6. Ordóñez N G. Application of mesothelin immunostaining in tumor diagnosis. Am J Surg Pathol. 2003; 27(11):1418-1428.

7. Hassan R, Laszik Z G, Lerner M, Raffeld M, Postier R, Brackett D. Mesothelin Is Overexpressed in Pancreaticobiliary Adenocarcinomas but Not in Normal Pancreas and Chronic Pancreatitis. Am J Clin Pathol. 2005; 124(6):838-845. doi:10.1309/F1B64CL7H8VJKEAF.

8. Parinyanitikul N, Blumenschein G R, Wu Y, et al. Mesothelin Expression and Survival Outcomes in Triple Receptor Negative Breast Cancer. Clin Breast Cancer. 2013; 13(5):378-384. doi:10.1016/j.clbc.2013.05.001.

9. Tchou J, Wang L-C, Selven B, et al. Mesothelin, a novel immunotherapy target for triple negative breast cancer. Breast Cancer Res Treat. 2012; 133(2):799-804. doi:10.1007/s10549-012-2018-4.

10. Kachala S S, Bograd A J, Villena-Vargas J, et al. Mesothelin Overexpression Is a Marker of Tumor Aggressiveness and Is Associated with Reduced Recurrence-Free and Overall Survival in Early-Stage Lung Adenocarcinoma. Clin Cancer Res. 2014; 20(4):1020-1028. doi:10.1158/1078-0432.CCR-13-1862.

11. Cheng W-F, Huang C-Y, Chang M-C, et al. High mesothelin correlates with chemoresistance and poor survival in epithelial ovarian carcinoma. Br J Cancer. 2009; 100(7):1144-1153. doi:10.1038/sj.bjc.6604964.

12. Hellstrom I, Friedman E, Verch T, et al. Anti-Mesothelin Antibodies and Circulating Mesothelin Relate to the Clinical State in Ovarian Cancer Patients. Cancer Epidemiol Biomarkers Prey. 2008; 17(6):1520-1526. doi:10.1158/1055-9965.EPI-08-0039.

13. Servais E L, Colovos C, Rodriguez L, et al. Mesothelin Overexpression Promotes Mesothelioma Cell Invasion and MMP-9 Secretion in an Orthotopic Mouse Model and in Epithelioid Pleural Mesothelioma Patients. Clin Cancer Res. 2012; 18(9):2478-2489. doi:10.1158/1078-0432.CCR-11-2614.

14. Chang M-C, Chen C-A, Chen P-J, et al. Mesothelin enhances invasion of ovarian cancer by inducing MMP-7 through MAPK/ERK and JNK pathways. Biochem J. 2012; 442(2):293-302. doi:10.1042/BJ20110282.

15. Bharadwaj U, Li M, Chen C, Yao Q. Mesothelin-Induced Pancreatic Cancer Cell Proliferation Involves Alteration of Cyclin E via Activation of Signal Transducer and Activator of Transcription Protein 3. Mol Cancer Res. 2008; 6(11):1755-1765. doi:10.1158/1541-7786.MCR-08-0095.

16. Chang M-C, Chen C-A, Hsieh C-Y, et al. Mesothelin inhibits paclitaxel-induced apoptosis through the PI3K pathway. Biochem J. 2009; 424(3):449-458. doi:10.1042/BJ20082196.

17. Wang L, Niu Z, Zhang L, et al. Clinicopathological significance of mesothelin expression in invasive breast cancer. J Int Med Res. 2012; 40(3):909-916.

18. Tozbikian G, Brogi E, Kadota K, et al. Mesothelin expression in triple negative breast carcinomas correlates significantly with basal-like phenotype, distant metastases and decreased survival. PloS One. 2014; 9(12):e114900.

19. Li Y R, Xian R R, Ziober A, et al. Mesothelin expression is associated with poor outcomes in breast cancer. Breast Cancer Res Treat. 2014; 147(3):675-684. doi:10.1007/s10549-014-3077-5.

20. Hassan R, Ho M. Mesothelin targeted cancer immunotherapy. Eur J Cancer. 2008; 44(1):46-53. doi:10.1016/j.ejca.2007.08.028.

21. Franko A, Dolzan V, Kovac V, Arneric N, Dodic-Fikfak M. Soluble mesothelin-related peptides levels in patients with malignant mesothelioma. Dis Markers. 2012; 32(2):123-131.

22. Scherpereel A, Grigoriu B, Conti M, et al. Soluble Mesothelin-related Peptides in the Diagnosis of Malignant Pleural Mesothelioma. Am J Respir Crit Care Med. 2006; 173(10):1155-1160. doi:10.1164/rccm.200511-17890C.

23. McIntosh M W, Drescher C, Karlan B, et al. Combining CA 125 and SMR serum markers for diagnosis and early detection of ovarian carcinoma. Gynecol Oncol. 2004; 95(1):9-15. doi:10.1016/j.ygyno.2004.07.039.

24. Hassan R. Detection and Quantitation of Serum Mesothelin, a Tumor Marker for Patients with Mesothelioma and Ovarian Cancer. Clin Cancer Res. 2006; 12(2):447-453. doi:10.1158/1078-0432.CCR-05-1477.

25. Prantner A M, Turini M, Kerfelec B, et al. Anti-Mesothelin Nanobodies for Both Conventional and Nanoparticle-Based Biomedical Applications. J Biomed Nanotechnol. 2015; 11(7):1201-1212.

26. Starodub A N, Ocean A J, Shah M A, et al. First-in-Human Trial of a Novel Anti-Trop-2 Antibody-SN-38 Conjugate, Sacituzumab Govitecan, for the Treatment of Diverse Metastatic Solid Tumors. Clin Cancer Res. 2015; 21(17):3870-3878. doi:10.1158/1078-0432.CCR-14-3321.

27. Nanda R, Chow L Q M, Dees E C, et al. Pembrolizumab in Patients With Advanced Triple-Negative Breast Cancer: Phase Ib KEYNOTE-012 Study. J Clin Oncol. 2016; 34(21):2460-2467. doi:10.1200/JCO.2015.64.8931.

28. Hassan R, Lerner M R, Benbrook D, et al. Antitumor activity of SS (dsFv) PE38 and SS1 (dsFv) PE38, recombinant antimesothelin immunotoxins against human gynecologic cancers grown in organotypic culture in vitro. Clin Cancer Res. 2002; 8(11):3520-3526.

29. LI Q, VERSCHRAEGEN C F, MENDOZA J, HASSAN R. Cytotoxic activity of the recombinant anti-mesothelin immunotoxin, SS1 (dsFv) PE38, towards tumor cell lines established from ascites of patients with peritoneal mesotheliomas. Anticancer Res. 2004; 24(3A): 1327-1336.

30. Hassan R, Bera T, Pastan I. Mesothelin A New Target for Immunotherapy. Clin Cancer Res. 2004; 10(12):3937-3942.

31. Hassan R, Broaddus V C, Wilson S, Liewehr D J, Zhang J. Anti Mesothelin Immunotoxin SS1P in Combination with Gemcitabine Results in Increased Activity against Mesothelin-Expressing Tumor Xenografts. Clin Cancer Res. 2007; 13(23):7166-7171. doi:10.1158/1078-0432.CCR-07-1592.

32. Zhang Y, Xiang L, Hassan R, Pastan I. Immunotoxin and Taxol synergy results from a decrease in shed mesothelin levels in the extracellular space of tumors. Proc Natl Acad Sci. 2007; 104(43):17099-17104.

33. Hassan R, Sharon E, Thomas A, et al. Phase 1 study of the antimesothelin immunotoxin SS1P in combination with pemetrexed and cisplatin for front-line therapy of pleural mesothelioma and correlation of tumor response with serum mesothelin, megakaryocyte potentiating factor, and cancer antigen: SS1P Plus Chemotherapy for Mesothelioma. Cancer. 2014; 120(21):3311-3319. doi:10.1002/cncr.28875.

34. Ho M, Hassan R, Zhang J, et al. Humoral immune response to mesothelin in mesothelioma and ovarian cancer patients. Clin Cancer Res. 2005; 11(10):3814-3820.

35. Le D T, Brockstedt D G, Nir-Paz R, et al. A Live-Attenuated Listeria Vaccine (ANZ-100) and a Live-Attenuated Listeria Vaccine Expressing Mesothelin (CRS-207) for Advanced Cancers: Phase I Studies of Safety and Immune Induction. Clin Cancer Res. 2012; 18(3):858-868. doi:10.1158/1078-0432.CCR-11-2121.

36. Sharon E, Zhang J, Hollevoet K, et al. Serum mesothelin and megakaryocyte potentiating factor in pancreatic and biliary cancers. Clin Chem Lab Med. 2012; 50(4). doi:10.1515/cclm.2011.816.

37. Kobayashi K, Sasaki T, Takenaka F, et al. A Novel PET Imaging Using 64 Cu-Labeled Monoclonal Antibody against Mesothelin Commonly Expressed on Cancer Cells. J Immunol Res. 2015; 2015:1-15. doi:10.1155/2015/268172.

38. Ter Weele E J, van Scheltinga A G T, Kosterink J G, et al. Imaging the distribution of an antibody-drug conjugate constituent targeting mesothelin with 89Zr and IRDye 800CW in mice bearing human pancreatic tumor xenografts. Oncotarget. 2015; 6(39):42081.

39. Tchouate Gainkam L O, Caveliers V, Devoogdt N, et al. Localization, mechanism and reduction of renal retention of technetium-99m labeled epidermal growth factor receptor-specific nanobody in mice. Contrast Media Mol Imaging. 2011; 6(2):85-92.

40. Eroles P, Bosch A, Alejandro Pérez-Fidalgo J, Lluch A. Molecular biology in breast cancer: Intrinsic subtypes and signaling pathways. Cancer Treat Rev. 2012; 38(6):698-707. doi:10.1016/j.ctrv.2011.11.005.

41. Keyaerts M, Xavier C, Heemskerk J, et al. Phase I Study of 68Ga-HER2-Nanobody for PET/CT Assessment of HER2 Expression in Breast Carcinoma. J Nucl Med. 2016; 57(1):27-33. doi:10.2967/jnumed.115.162024.

1. An anti-mesothelin single domain antibody which is labelled with a radionuclide wherein said single domain antibody i) binds to mesothelin with a dissociation constant (KD) of at least 5×10⁻⁸ and ii) cross-competes with the single domain antibody having the amino acid sequence SEQ ID NO: 1 for binding to mesothelin.

2. The single domain antibody of claim 1, wherein the single domain antibody binds to mesothelin with a dissociation constant (KD) of about 45 nM or less.

3. The single domain antibody of claim 1, wherein the single domain antibody comprises (a) a CDR1 having a sequence set forth as SEQ ID NO:2 (GIDLSLYR), (b) a CDR2 having a sequence set forth as SEQ ID NO:3 (ITDDGTS); and (c) a CDR3 having a sequence set forth as SEQ ID NO:4 (NAETPLSPVNY).

4. The single domain antibody of claim 1, wherein the single domain antibody comprises an amino acid sequence having at least 70% of identity with SEQ ID NO: 1.

5. The single domain antibody of claim 1, wherein the single domain antibody is humanized.

6. The single domain antibody of claim 1, wherein the single domain antibody is fused to a heterologous polypeptide.

7. The single domain antibody of claim 1 wherein the radionuclide is selected from the group consisting of γ-emitting and α-emitting radioisotopes and β-emitting radioisotopes, including but not limited to a radioisotope chosen from the group consisting of Actinium-225, Astatine-211, Bismuth-212, Bismuth-213, Caesium-137, Chromium-51, Cobalt-60, Cupper-64 Dysprosium-165, Erbium-169, Fermium-255, Fluor-18, Gallium-67, Gallium-68, Gold-198, Holmium-166, Indium-I11, Iodine-123, Iodine-124, Iodine-125, Iodine-131, Iridium-192, Iron-59, Lead-212, Lutetium-177, Molybdenum-99, Palladium-103, Phosphorus-32, Potassium-42, Rhenium-186, Rhenium-188, Samarium-153, Technetium-99m, Radium-223, Ruthenium-106, Sodium-24, Strontium-89, Terbium-149, Thorium-227, Xenon-133, Ytterbium-169, Ytterbium-177, and Yttrium-90.

8. A method of obtaining an image of a cancer in a subject in need thereof comprising i) administering to the subject a pharmaceutically acceptable composition comprising the radiolabeled single domain antibody of claim 1; ii) identifying a detectable signal from the radiolabeled single domain antibody in the subject and iii) generating an image of the detectable signal, thereby obtaining an image of the cancer in the subject.

9. The method of claim 8 wherein the signal is detected by Single-Photon Emission Computed Tomography (SPECT) or Positron Emission Tomography (PET).

10. The method of claim 9 wherein the radionuclide for SPECT is Technetium-99m or Iodine-123.

11. The method of claim 9 wherein the radionuclide for PET is Fluor-18 or Gallium-68.

12. A method of treating cancer in a patient in need thereof comprising administering to the subject a therapeutically effective amount of the radiolabeled single domain antibody of claim 1.

13. The method of claim 8 wherein the patient suffers from a cancer selected from the group consisting of mesothelioma, prostate cancer, lung cancer, stomach cancer, squamous cell carcinoma, pancreatic cancer, cholangiocarcinoma, breast cancer and ovarian cancer.

14. The method of claim 8 wherein the cancer is a metastatic cancer.

15. A pharmaceutical composition comprising the single domain antibody of claim 1.

16. The method of claim 12 wherein the patient suffers from a cancer selected from the group consisting of mesothelioma, prostate cancer, lung cancer, stomach cancer, squamous cell carcinoma, pancreatic cancer, cholangiocarcinoma, breast cancer and ovarian cancer.

17. The method of claim 12 wherein the cancer is a metastatic cancer.


SEQUENCE LISTING (4 sequences)

SEQ ID NO: 1 - protein, 116 aa, Lama glama
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 2 - protein, 8 aa, Artificial
  CDR1
GIDLSLYR

SEQ ID NO: 3 - protein, 7 aa, Artificial
  CDR2
ITDDGTS

SEQ ID NO: 4 - protein, 11 aa, Artificial
  CDR3
NAETPLSPVNY